(19) United States
(12) Patent Application Publication
(10) Pub. No.: US 2005/0147686 A1
(43) Pub. Date: Jul. 7, 2005

(54) Method for preparing a porous polymer structure

(76) Inventors: Dirk Wybe Grijpma, Hengelo (NL); Qingpu Hou, Hengelo (NL); Jan Feijen, Hengelo (NL)

Correspondence Address: THE WEBB LAW FIRM, P.C., 700 KOPPERS BUILDING, 436 SEVENTH AVENUE, PITTSBURGH, PA, 15219, US

(21) Appl. No.: 10/503,354

(22) PCT Filed: Jan. 23, 2003
(86) PCT No.: PCT/NL03/00050

(30) Foreign Application Priority Data
Feb. 1, 2002 (NL) 1019888

(51) Int. Cl. A61K 9/14; B01J 13/02

(52) U.S. Cl. 424/489; 264/4.1

(57) ABSTRACT

A polymer is dissolved in a first liquid or the polymer is brought into the liquid phase. To the solution are added particles which are insoluble in the first liquid, so that a suspension or dispersion results. The suspension or dispersion is then transferred into an excess of second liquid, in which both the polymer and the particles are insoluble. The second liquid is mixed vigorously and this results in a precipitate of the polymer with the particles encapsulated therein.

The present invention relates to a method for preparing porous polymer structures with homogeneously distributed cavities which can be thermally processed after moulding, particularly for the purpose of preparing polymer structures with a low glass point.

The present invention relates to a method for preparing a porous polymer structure. A polymer is dissolved in a first liquid or the polymer is brought into the liquid phase. To the solution are added particles which are insoluble in the first liquid, so that a suspension or dispersion results. The suspension or dispersion is then transferred into an excess of second liquid, in which both the polymer and the particles are insoluble. The second liquid is mixed vigorously and this results in a precipitate of the polymer with the particles encapsulated therein. This precipitate is then isolated, cut into granules and dried. These granules can be further processed in two ways. In the first method the granules are transferred into a third liquid in which the polymer does not dissolve but the particles do. The encapsulated particles are hereby leached from the polymer, whereby porous granules result which can be further processed. In the second method the granules are thermally processed and moulded. The moulded polymer with the particles encapsulated therein are placed in a fourth liquid in which the polymer does not dissolve but the particles do. The encapsulated particles are hereby leached from the moulded polymer, thereby resulting in a porous moulded polymer which can be further processed.

“Tissue engineering” is a relatively new development within medicine, wherein porous polymer matrices (“scaffolds”) are generally used as three-dimensional matrix for adhesion of cells and the formation of tissue in vitro and/or in vivo. Potential tissues and organs which can be prepared in this manner are for instance cartilage, bone, heart valves, nerves, muscles, bladder, liver and so on. A high degree of porosity is important for increasing the specific surface area for the cell adhesion and tissue growth.

Diverse methods are known for preparing porous polymer structures, such as for instance sintering, freeze-drying and phase inversion. WO 99/25391 and WO 01/10478 describe a method for preparing porous polymer structures using phase inversion. WO 99/25391 for instance describes a method for preparing porous polymer structures wherein a liquid polymer is mixed with particles, whereafter the mixture is frozen or gelled in order to obtain a stable encapsulation of the particles with polymer. Subsequent transfer of this frozen or gelled mixture into a liquid in which the polymer and the particles are not soluble results in a stable polymer matrix with the particles encapsulated therein. A porous polymer matrix is obtained by leaching the particles. A drawback of the above stated technique is that the form of the porous structure is predetermined with this technique, whereby thermal processing can no longer be carried out. A second drawback of this technique is that a porous structure can be obtained in which the cavities are not homogeneously distributed, since the particles settle during the phase inversion.

The object of the present invention is to provide an improved method for preparing porous polymer structures with homogeneously distributed cavities which can be thermally processed, particularly for the purpose of preparing polymer structures with a low glass point.

This object is achieved with the present invention by a method for preparing a porous polymer structure, comprising of:
(a) providing a first liquid with at least one polymer dissolved therein for the purpose of forming a first polymer liquid;
(b) adding to the first polymer liquid at least one type of particle, insoluble in the first polymer liquid, for the purpose of forming a homogeneous suspension or dispersion of particles in the first polymer liquid;
characterized by adding the suspension to an excess of second liquid in which both the polymer and the particles are insoluble, whereby a precipitate is formed in which the particles are homogeneously distributed.

It is surprising that the particles remain homogeneously distributed in the polymer precipitate during precipitation. This has the result that after precipitation of the polymer in the second liquid a structure is obtained with the particles therein encapsulated in stable manner and homogeneously distributed. Polymer precipitates with particles encapsulated therein can hereby also be obtained from polymers, such as polymers with a lower glass point.

This precipitate is then isolated, dried and processed into granules. These granules can be further processed using two methods. In the first method the granules are transferred to a third liquid in which the polymer does not dissolve, but the particles do. The encapsulated particles are hereby leached from the polymer, whereby porous granules result which can be further processed by for instance a thermal process. In the second method the granules with the particles encapsulated therein are thermally processed and moulded. Thermal processing of the granules allows application of the granules in a large number of standard moulding techniques. The moulded polymer with the particles encapsulated therein is placed in a fourth liquid in which the polymer does not dissolve, but the particles do. The encapsulated particles are hereby leached from the moulded polymer, thereby resulting in a porous moulded polymer which can be further processed or used immediately.

According to the method of the present invention, at least one polymer is chosen from the group comprising polyethers, polyesters, polycarbonates, copolymers and block copolymers such as for instance poly(D,L-lactide) (PDLLA), poly(ether ester) (PEOT/PBT), poly(ε-aprolacton) (PCL), poly(trimethylene carbonate) (PTMC) dissolved in at least one organic or inorganic solvent or the liquid polymer, preferably at room temperature in a 2-10% solution (w/v). At least one type of particle, chosen from the group comprising organic compounds, inorganic compounds, salts, polymers, lipids, proteins, sugars or compositions thereof (75-90% w/v) is added while stirring to the polymer solution, whereby a suspension or dispersion of the particles in the polymer solution is obtained.

The obtained suspension can then be precipitated slowly in an excess of second solution such as for instance ethanol, methanol, isopropanol, ether and water. Both the polymer and the particles are insoluble in this second solution.

The obtained fibrous precipitate of particles enclosed by polymer is dried and processed into small granules. The granules can be further processed using at least two methods. In the first method the granules are placed in a third liquid in which the polymer does not dissolve but the particles do, such as for instance water. This third liquid is changed several times in order to achieve complete leaching of the particles. The obtained porous granules can be further processed as a part of biomedical applications. Possible processing methods are compression moulding, injection moulding, extrusion and in-mould labelling. In the second method the obtained precipitate is brought into the desired form by means of at least one thermal process. Suitable thermal processes are for instance injection, extrusion, compression-moulding and in-mould labelling. The moulded polymer is then placed in a fourth liquid in which the polymer does not dissolve but the particles do, such as for instance water. This fourth liquid is changed several times in order to achieve complete leaching of the particles. The obtained porous moulded polymer structure can be further processed as part of biomedical applications.

A preferred method of the present invention relates to dissolving polymer in a 2-10% solution (w/v) in chloroform at room temperature. NaCl particles (75-90% w/v) are added while stirring to the polymer solution, wherein a homogeneous suspension of the salt in the polymer solution is obtained.

The obtained dispersion is then precipitated in a tenfold volume of ethanol.

The obtained fibrous polymer salt precipitate is dried for three days under vacuum and cut into small granules of about 4×4×4 mm. The granules are then brought into the desired form by means of compression moulding. The moulded polymer structure is incubated in water for leaching the NaCl particles for 3 days, while the water is continuously changed until complete leaching of the particles is achieved.

### SHORT DESCRIPTION OF THE FIGURES

FIG. 1

A schematic representation of the method of the present invention.

FIG. 2

Porous poly(D,L-lactide) (PDLLA) granules after leaching and drying.

FIG. 3

The result of compression moulding of the granules in a mould after leaching of the particles.

FIG. 4

The processing of the polymer salt precipitate by means of compression moulding.

FIG. 5

The leaching of salt-polymer structures moulded by means of compression moulding.

FIG. 6

Porosity of poly(D,L-lactide) (PDLLA) matrices shown as a function of the concentration of salt particles at different sizes.

FIG. 7

SEM micrograph of a poly(D,L-lactide) (PDLLA) matrix after precipitation, compression moulding and leaching. The porosity is 96% v/v and the particle size 250-425 μm.

FIG. 8

Porosity of 1000 PEOT70PBT30 matrices shown as a function of the concentration of salt particles at different sizes.

FIG. 9

SEM micrograph of a 1000 PEOT70PBT30 matrix after precipitation, compression moulding and leaching. The porosity is 93% v/v and the particle size 500-710 μm.

FIG. 10

Porosity of poly(e-aprolacton) (PCL) matrices shown as a function of the concentration of salt particles at different sizes.

FIG. 11

SEM micrograph of a poly(e-aprolacton) (PCL) matrix after precipitation, compression moulding and leaching. The porosity is 92% v/v and the particle size 106-250 μm.

FIG. 12

SEM micrograph of a poly(trimethylene carbonate) (PTMC) matrix. The porosity is 90% v/v and the particle size 106-250 μm.

### EXAMPLES

### Example 1

The method according to the invention was compared to two conventional methods for preparing porous polymer structures with diverse biodegradable polymers: (1) sintering and (2) mixing polymer powders with leachable salt particles, followed by “compression moulding” and leaching of the particles. The porosity of the prepared structures was determined on the basis of the volume and the weight of the porous structure and the densities of the solid polymers: PDLLA: 1.25 g/ml; 1000PEOT70PBT30: 1.10 g/ml; PCL: 1.10 g/ml.

(1) Sintering:

Polymer particles were prepared by granulating polymers at liquid nitrogen temperature in an IKA laboratory “grinder”. The polymer particles were sieved to different diameters, varying from 0-250 μm, 250-425 μm, 435-500 μm, 500-710 μm, 710-1000 μm and 1000-1180 μm. The sintering was carried out in cylindrical moulds (8 mm high, diameter 17 mm) on a hot press, at a pressure of 100 kPa. The sintering temperature was close to the glass temperature or the melting temperature of the polymer.

PDLLA

The sintering temperature was 55° C. for 1 hour. It was found that under these conditions the particle size represented the most important variable for the porosity. Table 1 illustrates this effect. The maximum porosity which could be obtained was about 60% by volume.

TABLE 1
Porosity of PDLLA matrices prepared by sintering

| polymer particle size (μm) | porosity (% by volume) |
|---|---|
| <250 | 58.2 |
| 250-425 | 48.2 |
| 425-500 | 42.9 |
| 500-710 | 42.7 |
| 710-1000 | 40.6 |
| 1000-1180 | 39.1 |

1000PEOT70PBT30

The sintering temperature was 115° C. for 2 hours. Under these conditions the particle size was the most important variable for determining the porosity. The maximum obtained porosity was about 70% by volume, as shown in table 2.

TABLE 2
Porosity of 1000PEOT70PBT30 matrices prepared by sintering

| polymer particle size (μm) | porosity (% by volume) |
|---|---|
| <250 | 71.5 |
| 250-425 | 50.6 |
| 425-500 | 56.4 |
| 500-710 | 51.8 |
| 710-1000 | 47.9 |
| 1000-1180 | 47.0 |

PCL

Porous PCL structures could not be prepared by means of sintering because the polymer could not be granulated, not even by lowering the temperature to −196° C.

(2) Compression Moulding of Salt/Polymer Mixtures and Leaching of the Salt

Polymer particles were prepared by granulating the polymer in an IKA grinder. The particles were sieved so as to obtain particles with diameters varying from 0-250 μm, 250-425 μm, 435-500 μm, 500-710 μm. NaCl salt particles were likewise sieved to diameters varying from 0-250 μm, 250-425 μm, 435-500 μm, 500-710 μm. The polymer and salt particles were mixed homogeneously in different ratios, varying from 60-90% w/v. Compression moulding was carried out in cylindrical moulds (8 mm high, diameter 17 mm) on a hot press at a pressure of 3.5 MPa. Leaching of the salt particles was carried out in an excess of demineralized water while stirring moderately. Optimal results were achieved when the polymer particle size is smaller than or equal to the salt particle size.

PDLLA:

Table 3 shows the stability and porosity obtained after compression moulding of-PDLLA/salt mixtures and leaching of the salt particles. At a salt content of less than 6% w/v, it was not possible to leach the salt particles out of the moulded composite. Nor was it possible to obtain stable porous structures when the salt content was 90% w/v.

The stability during leaching of the matrix is designated as:

++ no fragmentation; + “crumbling away” of a few polymer fragments; +/− “crumbling away” of several polymer fragments; − “crumbling away” of many polymer fragments; −− complete disintegration of the matrix.

TABLE 3
Porous PDLLA structure obtained by compression moulding of salt/
polymer particle mixture, followed by leaching of the salt particles.

| | salt | salt | stability and |
|---|---|---|---|
| polymer particle | particle | content | porosity (% by |
| size (μm) | size (μm) | (% w/v) | volume) |
| <250 | <250 | 90 | −− |
| <250 | 250-425 | 80 | ++, 79.3 |
| <250 | 250-425 | 90 | − |
| <250 | 425-500 | 90 | +/− |
| <250 | 500-710 | 70 | ++, 73.0 |
| <250 | 500-710 | 90 | +/− |
| 250-425 | 250-425 | 80 | ++, 81.2 |
| 250-425 | 250-425 | 90 | −− |
| 250-425 | 425-500 | 90 | − |
| 250-425 | 500-710 | 70 | ++, 70.1 |
| 250-425 | 500-710 | 90 | +/− |
| 425-500 | 425-500 | 90 | +/− |
| 425-500 | 500-710 | 90 | +/− |
| 500-710 | 500-710 | 70 | ++, 71.8 |
| 500-710 | 500-710 | 75 | ++, 77.2 |
| 500-710 | 500-710 | 80 | ++, 80.4 |
| 500-710 | 500-710 | 90 | − |

10000PEOT70PBT30

Table 4 shows the stability and porosity obtained after preparation of the porous 1000PEOT70PBT30 structure. The best results were obtained when the size of the polymer particles was smaller than or equal to the size of the salt particles. At a salt content of less than 60% w/v it was not possible to leach the salt particles out of the composite. Nor was it possible to obtain a stable porous structure at a salt content of 90% w/v.

TABLE 4
Porous 1000PEOT70PBT30 structure obtained by compression
moulding of salt/polymer particle mixture, followed by leaching of
the salt particles.

| polymer | salt | salt | stability and |
|---|---|---|---|
| particle size | particle | content | porosity |
| (μm) | size (μm) | (% w/v) | (% by volume) |
| <250 | 250-425 | 70 | ++ |
| <250 | 250-425 | 80 | ++ |
| <250 | 250-425 | 90 | +/− |
| 250-425 | 425-500 | 60 | ++ |
| 250-425 | 425-500 | 70 | ++ |
| 250-425 | 425-500 | 80 | ++ |
| 250-425 | 425-500 | 90 | − |
| 250-425 | 500-710 | 80 | ++ |
| 250-425 | 710-1000 | 80 | + |
| 425-500 | 500-710 | 60 | ++ |
| 425-500 | 500-710 | 70 | ++ |
| 425-500 | 500-710 | 80 | ++ |
| 425-500 | 500-710 | 90 | +/− |
| 425-500 | 710-1000 | 80 | + |
| 500-710 | 710-1000 | 60 | + |
| 500-710 | 710-1000 | 70 | +/− |
| 500-710 | 710-1000 | 80 | +/− |
| 500-710 | 710-1000 | 90 | −− |

PCL

Porous PCT structures could not be prepared in this manner because the polymer could not be granulated.

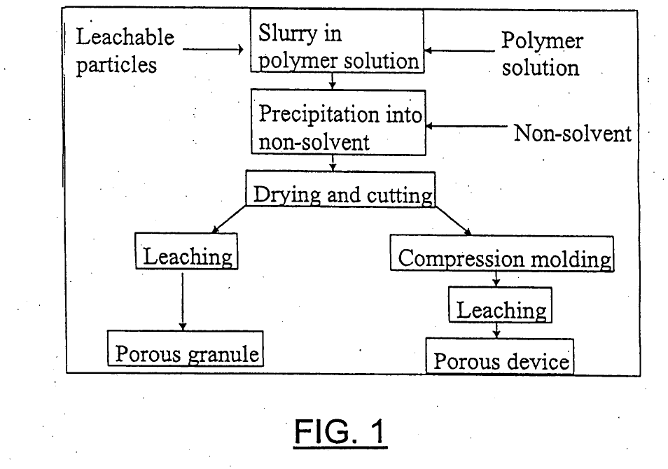

(3) Method According to the Invention (FIG. 1):

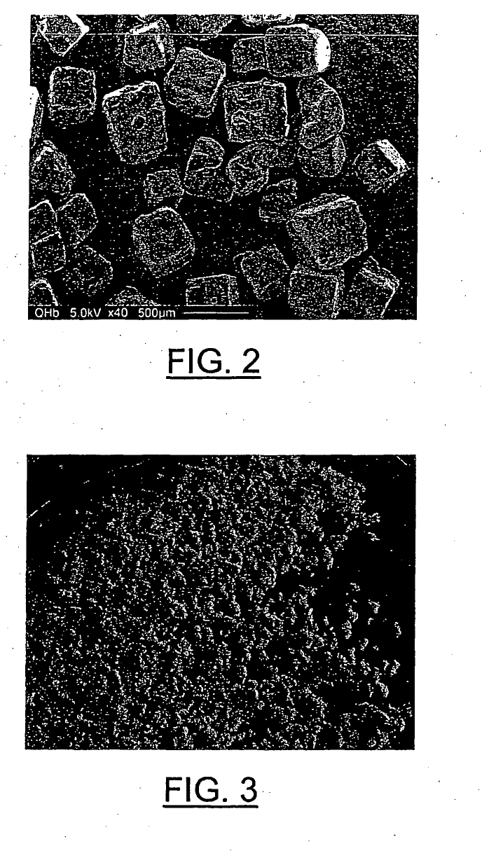

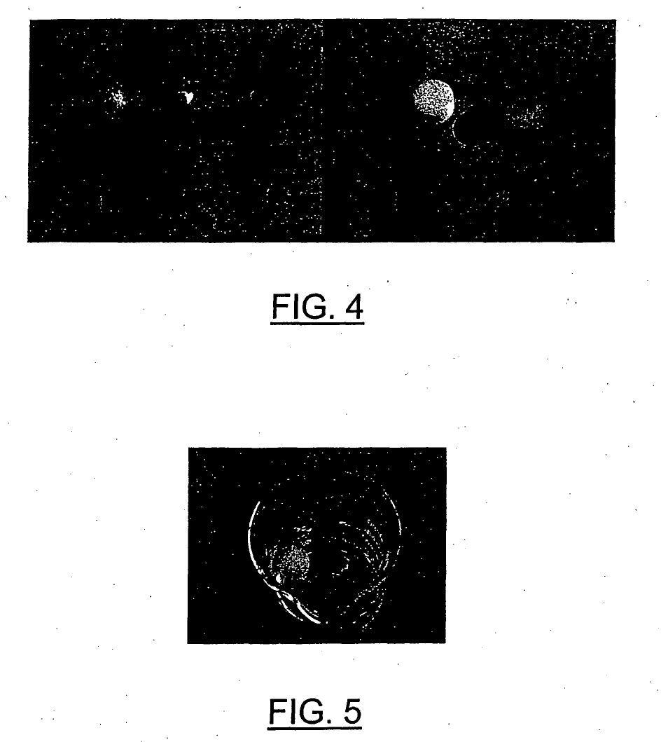

The polymers were dissolved in trichloromethane in a concentration of 10% w/v per vol %. Salt particles were added hereto in concentrations of 70-95% w/v. The salt was sieved in order to obtain particles with sizes varying from 106-250 μm, 250-425 μm, 425-500 μm, 500-710 μm (FIG. 2). The polymer-salt mixture was precipitated while stirring vigorously in a tenfold excess of a non-solvent. After drying and cutting into granules of 4×4×4 mm (FIG. 3), the precipitate was processed using compression moulding in a cylindrical mould of 8 mm×17 mm on a hot press at 3.5 Mpa (FIG. 4). After leaching the salt particles with water and drying the porous matrices, the porosity was determined. The leaching was carried out with demineralized water while stirring moderately (FIG. 5).

PDLLA

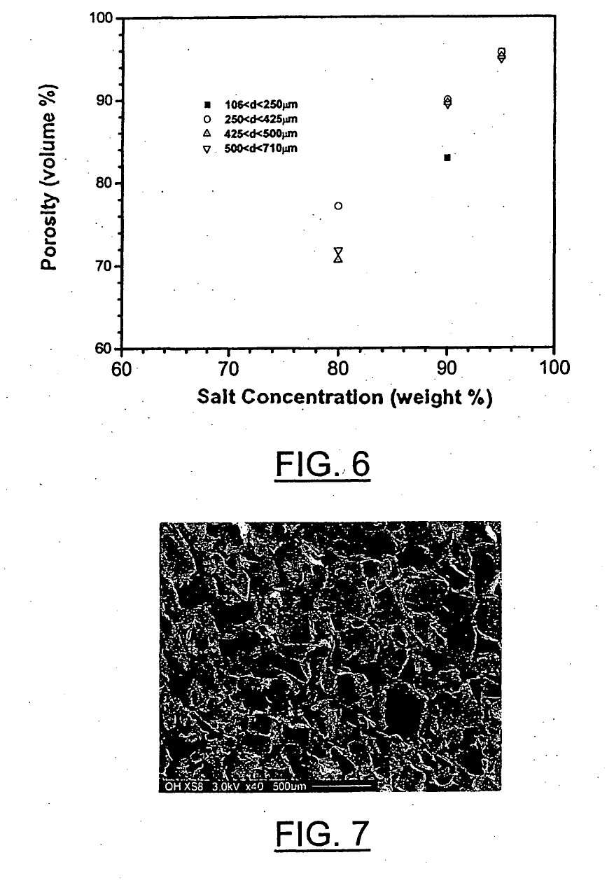

FIG. 6 shows the obtained volume porosity as a function of the salt content. The % w/v of salt was varied between 80 and 95%. The volume porosity after leaching was not strongly influenced by the size of the salt particles and the resulting pore dimensions. High porosity structures with considerably varying porosities and pore dimensions can be prepared in this manner.

FIG. 7 shows an SEM photograph of a PDLLA structure with a porosity of 60% by volume and pore dimensions of 250-425 μm. This shows that a regular structure was obtained with homogeneously distributed, mutually connected pores. Herein is shown that the pore size is comparable to the particle size of the salt particles. The porosity of the polymer structures can be controlled by variation of the salt concentration and the particle size.

1000PEOT70PBT30:

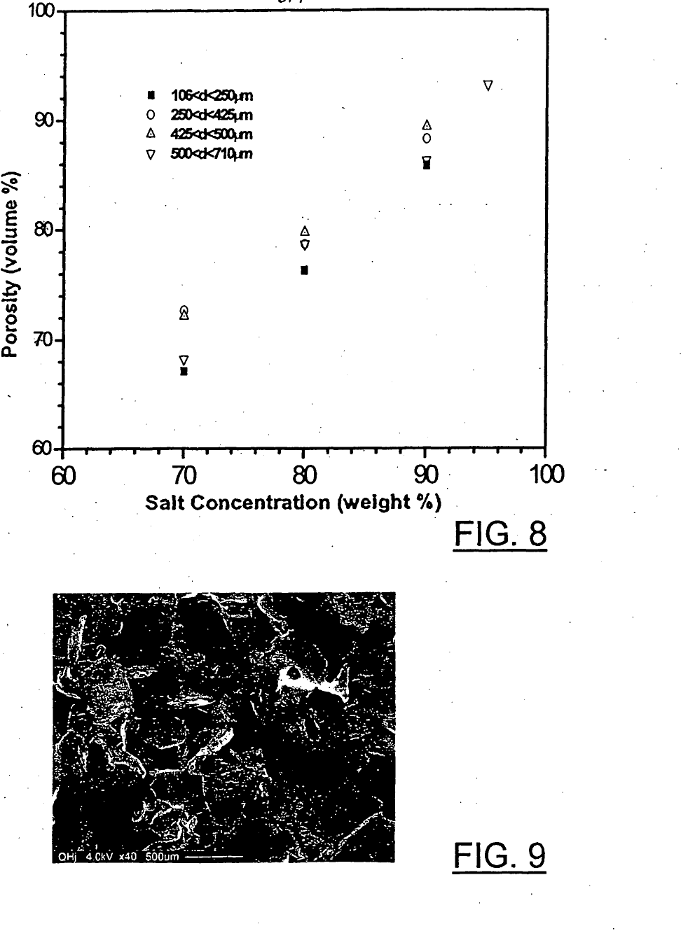

FIG. 8 shows the obtained volume porosity as a function of the salt concentration. The % w/v of salt was varied between 70 and 95%. The volume porosity after leaching was not strongly influenced by the size of the salt particles and the resulting pore dimensions. High porosity structures with considerably varying porosities and pore dimensions can be prepared in this manner.

FIG. 9 shows an SEM photograph of a 1000PEOT70PBT30 structure with a porosity of 95% by volume and pore dimensions of 500-710 μm. This shows that a regular structure was obtained with homogeneously distributed, mutually connected pores. The porosity of the polymer structures can be controlled by variation of the salt concentration and the particle size.

PCL:

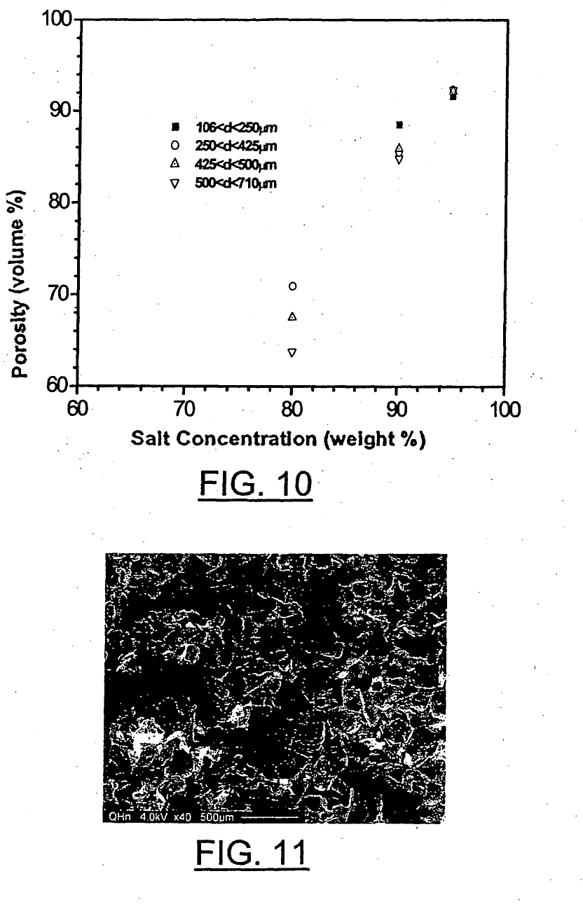

FIG. 10 shows the obtained volume porosity as a function of the salt concentration of PCL. The % w/v of salt was varied between 80 and 95%. The volume porosity after leaching was not strongly influenced by the size of the salt particles and the resulting pore dimensions. High porosity structures with considerably varying porosities and pore dimensions can be prepared in this manner.

FIG. 11 shows an SEM photograph of a PCL structure with a porosity of 92% by volume and pore dimensions of 106-250 μm. This shows that a regular structure was obtained with homogeneously distributed, mutually connected pores. The porosity of the polymer structures can be controlled by variation of the salt concentration and the particle size.

It will be apparent from the foregoing that polymer structures with high porosity (>90% by volume) with variable pore size can only be obtained using the method according to the invention. Porous PCL structures could only be prepared using the method according to the invention.

Sintering of polymer particles produces polymer structures with a relatively low porosity (up to 70% by volume). The method wherein polymer particles and salt particles are mixed, compression moulded and leached produces fragile structures which fragment during the leaching process when the salt content is greater than 90% w/v.

### Example 2

Preparation of Rubber-Like Porous Structures

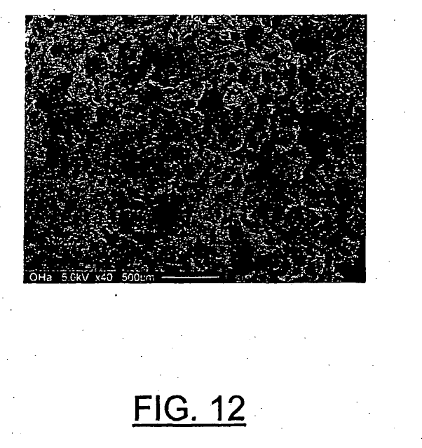

A porous rubber-like structure was prepared using the method according to the invention. For this purpose a poly(trimethylene carbonate) PTMC polymer with high molecular weight was dissolved in trichloromethane in a concentration of 2% (w/v). Salt particles of 106-250 μm were added to the solution and the mixture was precipitated in a tenfold excess of isopropanol while stirring vigorously. The precipitate was dried and washed in water. FIG. 12 shows the obtained porous structure.

1-28. (canceled)

29. A method for preparing a porous polymer structure, comprising:
a) providing a first liquid with at least one polymer dissolved therein for the purpose of forming a first polymer liquid;
b) adding to the first polymer liquid at least one type of particle, insoluble in the first polymer liquid, for the purpose of forming a suspension or dispersion of particles in the first polymer liquid;
wherein by adding the suspension or dispersion to an excess of second liquid in which both the polymer and the particles are insoluble for the purpose of forming a precipitate having particles encapsulated therein in homogeneous distribution.

30. The method as claimed in claim 29, wherein the formed precipitate is isolated from the second liquid.

31. The method as claimed in claim 30, wherein the precipitate is dried.

32. The method as claimed in claim 31, wherein the dried precipitate is processed into granules.

33. The method as claimed in claim 32, wherein the granules are transferred at least once into a third liquid.

34. The method as claimed in claim 33, wherein the third liquid is a solvent for the particles.

35. The method as claimed in claim 33, wherein the third liquid is not a solvent for the polymer.

36. The method as claimed in claim 33, wherein the third liquid is water.

37. The method as claimed in claim 33, wherein the granules are isolated from the third liquid so as to obtain porous granules.

38. The porous granules prepared with the method as claimed in claim 33.

39. The porous granules as claimed in claim 38, wherein a porosity of 60 to 99% v/v is obtained by varying the particle size and the quantity of particles.

40. The method as claimed in claim 31, wherein the granules and/or the precipitate are thermally processed and moulded.

41. The method as claimed in claim 40, wherein the thermal process is chosen from the group comprising injection, extrusion, compression moulding, in-mould labelling casting or combinations thereof.

42. The method as claimed in claim 40, wherein the moulded polymer structure is placed in at least a fourth liquid.

43. The method as claimed in claim 42, wherein the fourth liquid is a solvent for the particles.

44. The method as claimed in claim 42, wherein the fourth liquid is not a solvent for the polymer.

45. The method as claimed in claim 42, wherein the fourth liquid is water.

46. The method as claimed in claim 42, wherein the moulded polymer structure is isolated so as to obtain a porous moulded polymer structure.

47. A porous moulded polymer structure prepared according to the method of claim 42.

48. The porous moulded polymer structure as claimed in claim 47, wherein a porosity of 60 to 99% v/v is obtained by varying the particle size and the quantity of particles.

49. The method as claimed in claim 29, wherein the first liquid is chosen from the group comprising the liquid polymer, organic and inorganic liquids or compositions thereof.

50. The method as claimed in claim 29, wherein the particles are chosen from the group comprising organic compounds, inorganic compounds, salts, polymers, lipids, proteins, sugars or compositions thereof.

51. The method as claimed in claim 50, wherein the particles are NaCl crystals.

52. The method as claimed in claim 50, wherein the particles have a size of 0.001 to 5 mm.

53. The method as claimed in claim 29, wherein the second liquid is chosen from the group comprising ethanol, methanol, isopropanol, ether, water or compositions thereof.

54. A biomedical device prepared from the porous polymer structure as claimed in claim 47.

55. A biomedical device prepared from the porous granules as claimed in claim 38.

56. The method as claimed in claim 50, wherein the particles have a size of 0.1 to 1.5 mm.

57. The method as claimed in claim 50, wherein the particles have a size of 0.1 to 1 mm.
